(19) United States
(12) Patent Application Publication
(10) Pub. No.: US 2021/0374872 A1
(43) Pub. Date: Dec. 2, 2021

(54) VARIABLE COPAY SYSTEM

(71) Applicant: Syneos Health, LLC, Morrisville, NC (US)

(72) Inventors: Jeffrey J. Stewart, Cary, NC (US); Brian Cotten, Cary, NC (US)

(73) Assignee: Syneos Health, LLC, Morrisville, NC (US)

(21) Appl. No.: 17/333,151

(22) Filed: May 28, 2021

Related U.S. Application Data
(60) Provisional application No. 63/032,143, filed on May 29, 2020.

(51) Int. Cl. G06Q 40/08; G16H 20/10

(52) U.S. Cl. CPC G06Q 40/08; G16H 20/10

(57) ABSTRACT

A computing system receives an indication of a pharmaceutical to be prescribed to a patient. Based on the indication, the computing system receives benefit information from a payer system associated with the patient. The computing system receives one or more rules associated with a manufacturer system associated with the pharmaceutical to be prescribed to the patient. The computing system generates a variable copay program for the patient. The variable copay program includes a variable amount of copay assistance for each fill of the pharmaceutical to be prescribed to the patient. The computing system generates a table accessible to one or more third party systems, wherein the table comprises the variable copay program.

### CROSS-REFERENCE TO RELATED APPLICATIONS

This application claims priority to U.S. Application Ser. No. 63/032,143, filed May 29, 2020, which is hereby incorporated by reference in its entirety.

### FIELD OF THE DISCLOSURE

Embodiments disclosed herein generally relate to a variable copay assistant system and a method of operating the same.

### BACKGROUND

Prescription prices, even with insurance, can be a heavy burden on patients and manufacturers. For patients, the cost of a prescription may be so high that purchasing or continuing with a prescription can have major financial implications. For manufacturers, patients opting out of certain pharmaceuticals may lower profits or revenue.

### SUMMARY

In some embodiments, a method is disclosed herein. A computing system receives an indication of a pharmaceutical to be prescribed to a patient. Based on the indication, the computing system receives benefit information from a payer system associated with the patient. The benefit information includes one or more parameters for an insurance program provided by the payer system to the patient. The computing system receives one or more rules associated with a manufacturer system associated with the pharmaceutical to be prescribed to the patient. The one or more rules define conditions under which copay assistance is used to cover out-of-pocket responsibilities for the patient. The computing system generates a variable copay program for the patient. The variable copay program includes a variable amount of copay assistance for each fill of the pharmaceutical to be prescribed to the patient. The computing system generates a table accessible to one or more third party systems, wherein the table comprises the variable copay program.

In some embodiments, a non-transitory computer readable medium is disclosed herein. The non-transitory computer readable medium has one or more sequences of instructions, which, when executed by one or more processors, causes a computing system to perform operations. The operations include receiving, by the computing system, an indication of a pharmaceutical to be prescribed to a patient. The operations further include, based on the indication, receiving, by the computing system, benefit information from a payer system associated with the patient. The benefit information includes one or more parameters for an insurance program provided by the payer system to the patient. The operations further include receiving, by the computing system, one or more rules associated with a manufacturer system associated with the pharmaceutical to be prescribed to the patient. The one or more rules define conditions under which copay assistance is used to cover out-of-pocket responsibilities for the patient. The operations further include generating, by the computing system, a variable copay program for the patient. The variable copay program includes a variable amount of copay assistance for each fill of the pharmaceutical to be prescribed to the patient. The operations further include generating, by the computing system, a table accessible to one or more third party systems, wherein the table comprises the variable copay program.

In some embodiments, a system is disclosed herein. The system includes one or more processors and a memory. The memory has programming instructions stored thereon, which, when executed by the one or more processors, causes the system to perform operations. The operations include receiving an indication of a pharmaceutical to be prescribed to a patient. The operations further include, based on the indication, receiving benefit information from a payer system associated with the patient. The benefit information includes one or more parameters for an insurance program provided by the payer system to the patient. The operations further include receiving one or more rules associated with a manufacturer system associated with the pharmaceutical to be prescribed to the patient. The one or more rules define conditions under which copay assistance is used to cover out-of-pocket responsibilities for the patient. The operations further include generating a variable copay program for the patient. The variable copay program includes a variable amount of copay assistance for each fill of the pharmaceutical to be prescribed to the patient. The operations further include generating a table accessible to one or more third party systems, wherein the table comprises the variable copay program.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrated only typical embodiments of this disclosure and are therefore not to be considered limiting of its scope, for the disclosure may admit to other equally effective embodiments.

FIG. 1 is a block diagram illustrating a computing environment, according to one exemplary embodiment.

FIG. 2 is a block diagram illustrating an exemplary workflow, according to example embodiments.

FIG. 3 is a block diagram illustrating an exemplary workflow, according to example embodiments.

FIG. 4 is a flow diagram illustrating a method of generating a variable copay plan for a patient, according to example embodiments.

FIG. 5 is a chart that illustrates a first fill abandonment rate, according to example embodiments.

FIG. 6 is a chart that illustrates a pharmaceutical half-life, according to example embodiments.

FIG. 7 is a chart that illustrates average patient days on therapy, according to example embodiments.

FIG. 8A illustrates a system bus computing system architecture, according to example embodiments.

FIG. 8B illustrates a computer system having a chipset architecture, according to example embodiments.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

Patients are typically required by their insurance company to pay all or part of the cost of a pharmaceutical. These payments typically come in the form of copays (e.g., flat price) or co-insurance (e.g., a percentage of the price of the drug, which may be substantially higher than a copay). In addition, insurance plans may also have deductibles that can run several thousands of dollars. Until a patient reaches the deductible amount, the patient may be required to pay the full cost of the pharmaceutical. Additionally, there may also be an out-of-pocket maximum. After patients reach the out-of-pocket maximum, they may pay nothing or a de minimis amount for each prescription or pharmaceutical.

Generally, insurance plans may fall into two categories: high deductible health plans (HDHP) and non-HDHPs. The HDHPs may have deductibles that average more than double the non-HDHPs. HDHPs may thus have a high cost burden to the patient, especially at the beginning of a calendar year before the deductible is paid down.

As those skilled in the art recognize, patients are price sensitive. When patients are asked to pay higher dollar amounts, they may not ultimately fill the prescription recommended by physicians. As such, there is a constant battle to find ways to continue to engage the patient, even when costs associated with a pharmaceutical are high.

One or more techniques described herein provides a system that is able to dynamically adapt to patients' needs, while achieving a higher profit for manufacturers than what is currently available in the marketplace. Such approach may be referred to as a variable copay approach, by which copay assistance may be available for a plurality of insurance benefits that include at least one rule for a first insurance benefit and at least one rule for a second insurance benefit.

The term “user” as used herein includes, for example, a person or entity that owns a computing device or wireless device; a person or entity that operates or utilizes a computing device; or a person or entity that is otherwise associated with a computing device or wireless device. It is contemplated that the term “user” is not intended to be limiting and may include various examples beyond those described.

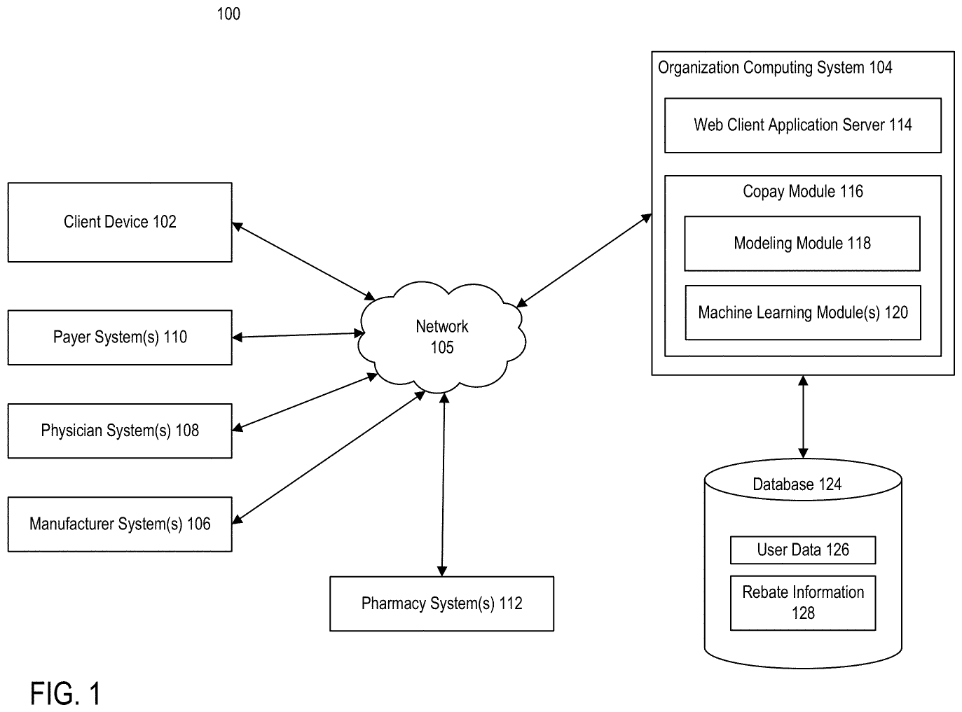

FIG. 1 is a block diagram illustrating a computing environment 100, according to one embodiment. Computing environment 100 may include at least one or more client devices 102, an organization computing system 104, one or more manufacturer systems 106, one or more physician systems 108, and one or more payer systems 110 communicating via network 105.

Network 105 may be representative of any suitable type, including individual connections via the Internet, such as cellular or Wi-Fi networks. In some embodiments, network 105 may connect terminals, services, and mobile devices using direct connections, such as radio frequency identification (RFID), near-field communication (NFC), Bluetooth™, low-energy Bluetooth™ (BLE), Wi-Fi™, ZigBee™, ambient backscatter communication (ABC) protocols, USB, WAN, or LAN. Because the information transmitted may be personal or confidential, security concerns may dictate one or more of these types of connection be encrypted or otherwise secured. In some embodiments, however, the information being transmitted may be less personal, and therefore, the network connections may be selected for convenience over security.

Network 105 may include any type of computer networking arrangement used to exchange data. For example, network 105 may be representative of the Internet, a private data network, virtual private network using a public network and/or other suitable connection(s) that enables components in computing environment 100 to send and receiving information between the components of computing environment 100.

Client device 102 may be operated by a user. For example, client device 102 may be a mobile device, a tablet, a desktop computer, or any computing system having the capabilities described herein. In some embodiments, client device 102 may be representative of a physical copay card, a virtual copay card, a physical check for copay assistance, an electronic check for copay benefits, and the like. In some embodiments, client device 102 may be configured to present a virtual card or quick-read (QR) code that allows a pharmacist to see if the user has secondary coverage for out of pocket expenses. Client device 102 may belong to or be provided to a patient or a customer. Patients or customers may include individuals such as, for example, those that may obtain a prescription commercially offered by a manufacturer and prescribed by a physician.

Payer system(s) 110 may be associated with an insurance company. For example, each payer system 110 may be associated with a health insurance provider (e.g., Anthem) or a pharmacy benefit management organization (e.g., Express Scripts). Generally, payer systems 110 may be representative of an entity associated with each patient or user, whereby the respective payer system 110 handles or manages a health care plan for the patient or user. In some embodiments, payer system 110 may offer the patient or user a HDHP or a non-HDHP. The HDHPs typically have high deductibles that average more than double the non-HDHPs and, thus, have higher cost burdens to the patient, especially at the beginning of the calendar year before the deductible may have been paid down. In both plans—HDHP and non-HDHP—the patient or user typically has various benefits. Exemplary benefits may include, but are not limited to, an out-of-pocket maximum, a deductible, a copay or coinsurance, and/or a monthly out-of-pocket. In some embodiments, these benefits may differ across patients or users, depending on the type of plan the patient or user has subscribed to. For example, a deductible may be high in the HDHP plan (e.g., $1500), may be low in a non-HDHP plan (e.g., $50), and still may be lower in other non-HDHP plans (e.g., $15). As such, healthcare plans across payer systems 110 may be variable.

Physician systems 108 may be associated with one or more physicians or physician offices. Physician systems 108 may be configured to communicate with one or more payer systems 110. For example, when processing a procedure, physician system 108 may communicate with a payer system 110 to determine whether a patient or user is eligible to undergo such procedure, i.e., whether it is covered by the patient's insurance. Physician systems 108 may also communicate with one or more pharmacy systems 112. For example, when prescribing an individual a prescription or pharmaceutical, physician system 108 may call in or communicate with a pharmacy system 112 so that an associated pharmacy may begin processing the prescription. Alternatively or additionally, in some embodiments, a physician may provide a patient or user with a physical script for a prescription, which the patient or user may take to a pharmacy to fill.

Pharmacy systems 112 may be associated with one or more pharmacies. Each pharmacy system 112 may be configured to communicate with one or more physician systems 108, one or more payer systems 110, and/or manufacturer systems 106. Pharmacy system 112 may be configured to process a script or prescription generated by a physician or physician system 108 and provide that prescription or pharmaceutical to a patient or user. In some embodiments, pharmacy system 112 may communicate with one or more payer systems 110 to determine how much the patient owes for this prescription. Generally, the amount owed by the patient may depend on their insurance plan with payer system 110. For example, the amount owed by the patient may depend on one or more of their out-of-pocket maximum, deductible, copay or coinsurance, and/or a monthly out-of-pocket.

Manufacturer systems 106 may be associated with one or more pharmaceutical manufacturers. For example, manufacturer systems 106 may be associated with one or more pharmaceuticals prescribed by a physician and used by a patient or user. In some embodiments, it may be beneficial for a manufacturer to pay for part of a patient's prescribed pharmaceutical. For example, copay assistance may be used to engage patients that may be price sensitive. Copay assistance is when a pharmaceutical manufacturer (e.g., manufacturer system 106) pays for a portion or all of the patient's cost burden. Copay assistance may be a substantial financial benefit to patients using this assistance. However, there generally is no means testing. Manufacturers are financially incentivized to provide copay assistance so that patients fill the prescription. Of course, if a patient is in the deductible period, the cost burden may be high, and the pharmaceutical company may have no profit or a negative profit when paying for these patients. In contrast, after the deductible period, paying the patient's out of pocket burden may substantially increase revenue and profits.

Profitability of copay assistance depends, in large part, on how much remains on a patient's deductible. In the beginning of the calendar year, copay assistance may be unprofitable for all patients while the deductible remains to be paid. That unprofitable period is likely to be longer for patients on HDHPs. For example, if an HDHP were receiving generous copay assistance for a $400 drug, the manufacturer could be paying the entire price of the drug on behalf of the patient through October and only have profitable prescriptions in November and December if the patient were still taking the drug. In contrast, the same copay assistance for a non-HDHP patient could switch to profitable prescriptions by June.

To account for this, manufacturer systems 106 may leverage functionality of organization computing system 104 to dynamically generate variable copay assistance that may be individually tailored to each patient.

Organization computing system 104 may be configured to communicate with one or more manufacturer systems 106. In some embodiments, organization computing system 104 may be associated with a healthcare company or clinical research company, such as Syneos Health, headquartered in Morrisville, N.C. Organization computing system 104 may include copay module 116. Copay module 116 may be comprised of one or more software modules. The one or more software modules may be collections of code or instructions stored on a media (e.g., memory of organization computing system 104) that represent a series of machine instructions (e.g., program code) that implements one or more algorithmic steps. Such machine instructions may be the actual computer code the processor of organization computing system 104 interprets to implement the instructions or, alternatively, may be a higher level of coding of the instructions that is interpreted to obtain the actual computer code. The one or more software modules may also include one or more hardware components. One or more aspects of an example algorithm may be performed by the hardware components (e.g., circuitry) itself, rather as a result of an instruction.

Copay module 116 may be configured to generate a variable copay plan for a patient for a pharmaceutical. Such optimized variable copay assistance may provide a higher profit to manufacturers than is currently available in the marketplace. Copay module 116 may include modeling module 118 and one or more machine learning modules 120.

Modeling module 118 may be configured to model or estimate various financial outcomes of possible copay plans for a patient for a given pharmaceutical. For example, copay assistant may be designed with various rules. These rules may define the conditions under which copay assistance may pay for a patient's out-of-pocket responsibilities. Exemplary rules may include, but are not limited to, pay as little as, annual cap, monthly cap, per-prescription cap, bridge discount, bridge discount number, and cash discount. Pay as little as may refer to the patient cost burden assuming other business rules are met. Annual cap may refer to a maximum benefit paid in a calendar year. Monthly cap may refer to a maximum benefit paid per prescription. Bridge discount may refer to a discount while patient insurance coverage is being investigated. For example, a bridge discount may be a free drug program for launch of new drugs to cover for initial lack of insurance coverage. The bridge discount may also be a program that provides drugs only to insurance plans where the drug may be covered generally, but where the claim is initially rejected while the prior authorization is completed. Cash discount may refer to a discount paid for patients without insurance covering the drug and where the patient is required to pay cash. Modeling module 118 may choose a given rule based on the market or based on an optimization target.

Modeling module 118 may be configured to optimize the various rules for any drug and insurance combination. To do so, modeling module 118 may take a multi-step approach. In some embodiments, modeling module 118 may be configured to estimate the patient out-of-pocket sensitivity for a first prescription fill for a pharmaceutical. In some embodiments, modeling module 118 may be configured to estimate patient out-of-pocket sensitivity for refills for the drug. In some embodiments, modeling module 118 may further model patient insurance. For example, modeling module 118 may take into consideration one or more of the patient's deductible, out-of-pocket maximum, copay or coinsurance, and/or patient out-of-pocket monthly fees for other prescriptions. In some embodiments, modeling module 118 may further model other financial characteristics for the manufacturer. Exemplary financial characteristics may include, but are not limited to, drug list price (e.g., wholesale acquisition cost), cost of goods sold, distribution costs, copay assistance administrative costs, rebate paid to an insurance company, 340B discount, and the like. In some embodiments, modeling module 118 may further model new-patient prescriptions. For example, modeling module 118 may model new-patient prescriptions each calendar month over a year or more. In some embodiments, modeling module 118 may test various combinations of rules. For example, depending on manufacturer input, modeling module 118 may model to optimize for revenue or optimize for profit.

As those skilled in the art recognize, different insurance types (e.g., HDHPs v. non-HDHPs) may have different optimal business rules. For example, a non-HDHP may have an optimal net profit with generous copay assistance (e.g., a patient may pay nothing for an entire calendar year). In another example, an HDHP patient may be optimized with less generous copay or no copay assistance for the same pharmaceutical.

Because copay assistance is often a compromise, modeling module 118 may optimize the business rules for the weighted average combination of insurance coverage of the anticipated patient. Modeling module 118 may generate copay assistance models to expose negative-profit HDHP patients to high enough out-of-profit costs so that these patients may discontinue the pharmaceutical while retaining profitable non-HDHP patients.

To do so, modeling module 118 may be configured to identify which business rule to apply based on a plurality of factors. For example, modeling module 118 may estimate a out-of-pocket sensitivity for the patient. In some embodiments, the out-of-pocket sensitivity may be a first fill out-of-pocket sensitivity (i.e., a first sensitivity) or a second fill out-of-pocket sensitivity (i.e., second sensitivity). Using this information, as well as the patient's insurance benefit information, modeling module 118 may generate or estimate the patient's pre-assistance out-of-pocket cost. The pre-assistance out-of-pocket cost may represent the patient's out-of-pocket cost without any copay assistance. The pre-assistance out-of-pocket cost may be based on one or more of the patient's deductible, out-of-pocket maximum, copay/co-insurance, out-of-pocket monthly expenses for other pharmaceuticals, and the like. Modeling module 118 may then apply a first business rule to determine or estimate a financial return, if copay assistance is offered. For example, modeling module 118 may apply one or more of pay as little as, annual cap, monthly cap, per-prescription cap, bridge discount, bridge number, cash discount, and the like. Using the first business rule, modeling module 118 may estimate a patient's post-assistant out-of-pocket cost. The post-assistance out-of-pocket cost may correspond to the patient's financial burden with copay assistance. The post-assistance out-of-pocket cost may be based on one or more of the proposed copay assistance, the patient's deductible, out-of-pocket maximum, copay/co-insurance, out-of-pocket monthly expenses for other pharmaceuticals, and the like. Modeling module 118 may then compare the out-of-pocket sensitivity to the post-assistance out-of-pocket cost to determine or estimate the probability of the patient paying the post-assistance out-of-pocket cost. Based on this information, modeling module 118 may project or estimate a financial return to manufacturer system 106 for offering copay assistance.

In some embodiments, modeling module 118 may repeat the above process for more than one business rule. In this manner, modeling module 118 may generate a plurality of financial return estimates or projections. Modeling module 118 may identify or choose that business rule that optimizes or returns the greatest financial return to manufacturer system 106.

Once modeled, modeling module 118 may generate a table of insurance benefits. The table may include at least one insurance benefit identifier and at least one benefit. In some embodiments, the at least one insurance benefit identifier may be selected from a list that includes a plan name, a payer name, a bank identification number, a plan ID, a group ID, and the like. In some embodiments the at least one benefit may be selected from a list that includes deductible, out-of-pocket maximum, copay, and coinsurance. In some embodiments, the table may include a manufacturer rebate. In some embodiments, the table may include a manufacturer discount.

Because some insurances may have different deductibles, modeling module 118 may provide a copay program for a plurality of benefits, with each benefit including at least one business rule for a first insurance benefit having a first deductible and at least one business rule for a second insurance benefit having a second deductible. In some embodiments, copay assistance for patients with different deductibles may be more profitable when the copay assistance is zero for at least one insurance benefit. Accordingly, in some embodiments, a copay program for a plurality of insurance benefits may be generated, where there is at least one business rule for a first insurance benefit having a first deductible where the business rule is zero benefit and at least one business rule for a second insurance benefit having a second deductible where the business rule is a benefit other than zero.

In some embodiments, variable copay programs for patients with different deductibles when the copay assistance is zero for at least one insurance benefit may be more profitable when zero is the benefit for the higher deductible. Accordingly, in some embodiments, modeling module 118 may generate a copay program for a plurality of insurance benefits that include at least one business rule for a first insurance benefit having a first deductible where the business rule may be zero benefit and at least one business rule for a second insurance benefit having a second deductible, where the business rule may be a benefit other than zero and where the first deductible may be greater than the second deductible.

In some embodiments, variable copay programs may be more profitable when different insurance benefits have different out-of-pocket maximum amounts. Accordingly, in some embodiments, modeling module 118 may generate a copay program for a plurality of insurance benefits that may include at least one business rule for a first insurance benefit having a first out-of-pocket maximum and at least one business rule for a second insurance benefit having a second out-of-pocket maximum.

In some embodiments, variable copay programs may be more profitable when different insurance benefits may have different copays. Accordingly, in some embodiments, modeling module 118 may generate a copay program for a plurality of insurance benefits that include at least one business rule for a first insurance benefit having a first copay and at least one business rule for a second insurance benefit having a second copay.

In some embodiments, variable copay programs may be more profitable when different insurance benefits have different coinsurance. Accordingly, in some embodiments, modeling module 118 may generate a copay program for a plurality of insurance benefits that may include at least one business rule for a first insurance benefit having a first coinsurance and at least one business rule for a second insurance benefit having a second coinsurance.

To generate a copay program, modeling module 118 may access user information in database 124. Database 124 may be configured to store user data 126. User data 126 may correspond to data associated with various patients. Such user data 126 may include, but is not limited to, information about each user's insurance program (if any) and information about each user's prescriptions and/or prescription history. In this manner, organization computing system 104 may have access information for generating a copay program for each patient.

In some embodiments, database 124 may further store rebate information 128. Rebate information 128 may correspond to rebate data between a manufacturer and a payer. For example, a manufacturer may offer a rebate for a pharmaceutical manufactured by the manufacturer.

In some embodiments, copay module 116 may leverage one or more machine learning modules 120 to generate a copay program for a given patient. Each of the one or more machine learning modules 120 may train a machine learning model to generate a customized or tailored copay program for each patient. Exemplary machine learning models may take the form of one or more supervised and/or unsupervised models. For example, exemplary machine learning models or algorithms may include, but are not limited to, random forest model, support vector machines, neural networks, clustering models, deep learning models, Bayesian algorithms, reinforcement learning models, and the like.

Each machine learning module 120 may be optimized or trained to generate a copay program that achieves a certain business rule. For example, machine learning module 120 may be configured to optimize the various rules for any drug and insurance combination. Using a specific example, for a given patient and a given pharmaceutical, machine learning module 120 may generate a first copay program that takes into account one or more of (1) when in the year the patient is starting the pharmaceutical; (2) the patient's deductible; (3) the patient's out-of-pocket maximum; (4) any coinsurance; and (5) any other out-of-pocket expenses for other pharmaceuticals. In some embodiments, machine learning module 120 may further take into account one or more financial characteristics, such as, but not limited to, the list price of the pharmaceutical, cost of goods sold, distribution costs, administrative costs, rebates paid to the patient's payer, and the like.

In some embodiments, manufacturer system 106 may adjust a target goal of machine learning module 120. For example, manufacturer system 106 may specify that machine learning module 120 should generate a copay program that optimizes for revenue. In this manner, machine learning module 120 may adjust one or more weights of a machine learning model to optimize for revenue. Using another example, manufacturer system 106 may specify that machine learning module 120 should generate a copay program that optimizes for profit. In this manner, machine learning module 120 may adjust one or more weights of a machine learning model to optimize for profit.

In some embodiments, machine learning modules 120 may utilize a brute force approach to determine those business rules that may be most applicable to a given patient.

Accordingly, machine learning modules 120 may leverage patient insurance information, patient pharmaceutical history information, one or more business rules, one or more financial characteristics, and one or more target goals of manufacturer system 106 to tailor a copay program to a specific patient.

In some embodiments, computing environment 100 may further include one or more third party systems. The one or more third party systems may be representative of an entity that offers deals or discounts to a user or patient. For example, a third party system may be representative of RelayHealth that provides deal with various pharmacies and/or payer systems that assist a user or patient with out of pocket pharmaceutical expenses. In operation, when a patient comes in with a written or electronic prescription, pharmacy system 112 may first sends the coverage request to the one or more third party systems. If, for example, there is a patient out-of-pocket expected, the one or more third party systems may pay a portion of or all of the out-of-pocket expense on the patient's behalf. The patient may be unaware that this has happened.

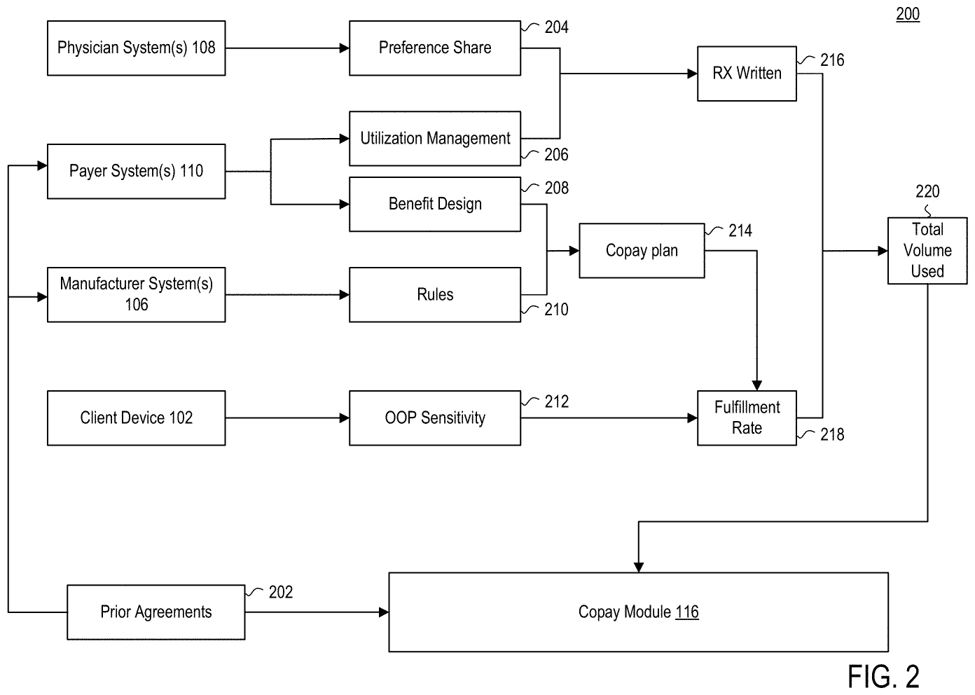

FIG. 2 is a block diagram illustrating an exemplary workflow 200, according to example embodiments. As shown, workflow 200 may start with physician system 108, or more broadly, a healthcare provider system. Physician system 108 may want to prescribe a pharmaceutical to a patient (“Preference Share”—block 204). When determining whether to prescribe a pharmaceutical to a patient, physician system 108 may take into account any prior authorization and/or step edits (“Utilization Management”—block 206). Based on the prior authorization and/or step edits, physician system 108 may generate a prescription (“RX Written”—block 216).

Turning to payer system 110, payer system 110 may take into account those items in block 206. For example, payer system 110 may have to pre-authorize a pharmaceutical before a physician can prescribe the pharmaceutical. Payer system 110 may further consider the design of the patient's insurance benefits (“Benefit Design”—block 208). For example, payer system 110 may consider whether the payer will allow the patient to gain access to the pharmaceutical with the patient's insurance plan. Payer system 110 may consider, for example, the copay amount, the patient's deductible, out-of-pocket maximum, and the like. This information may be provided to copay module 116 for modeling.

Referring to manufacturer system 106, at block 210, manufacturer system 106 may identify one or more rules. These rules may define the conditions under which copay assistance may pay for a patient's out-of-pocket responsibilities. Exemplary rules may include, but are not limited to, pay as little as, annual cap, monthly cap, per-prescription cap, bridge discount, bridge discount number, and cash discount. The rules at block 210 may be provided to copay module 116 for modeling.

Referring to client device 102, at block 212, a patient or user associated with client device 102 may have an out-of-pocket sensitivity. In some embodiments, the out-of-pocket sensitivity may depend on whether this is an initial script or prescription for a pharmaceutical or if this is a refill for the pharmaceutical. For example, for a first fill, patients typically exhibit higher price sensitivities at lower out-of-pocket levels when starting therapy.

Copay module 116 may generate a copay plan (block 214) for a pharmaceutical based on the out-of-pocket sensitivity of the patient (block 212), the rules of manufacturer system 106 (block 210), and the insurance benefit design (“Benefit Design”—block 208). In some embodiments, copay module 116 may further take into consideration any prior agreements (block 202) between the manufacturer and the payer. For example, if the manufacturer offers a rebate on a certain pharmaceutical, copay module 116 may take the rebate amount into consideration.

To generate the patient journey, copay module 116 may identify a start date for treatment. For example, copay module 116 may determine which month the user is filling the first script. Such information may assist copay module 116 in determining how the current pharmaceutical may affect the patient's deductible, out-of-pocket maximum, copay, and/or monthly out-of-pocket costs. In some embodiments, copay module 116 may generate a copay plan for the user via modeling module 118. For example, modeling module 118 may apply one or more algorithms to at least the data obtained from blocks 208-212, as well as any prior agreements between manufacturer and payer, to generate a variable copay plan for the patient.

For example, modeling module 118 may identify which business rule to apply based on a plurality of factors. For example, modeling module 118 may estimate an out-of-pocket sensitivity for the patient. In some embodiments, the out-of-pocket sensitivity may be a first fill out-of-pocket sensitivity (i.e., a first sensitivity) or a second fill out-of-pocket sensitivity (i.e., second sensitivity). Using this information, as well as the patient's insurance benefit information, modeling module 118 may generate or estimate the patient's pre-assistance out-of-pocket cost. The pre-assistance out-of-pocket cost may represent the patient's out-of-pocket cost without any copay assistance. The pre-assistance out-of-pocket cost may be based on one or more of the patient's deductible, out-of-pocket maximum, copay/co-insurance, out-of-pocket monthly expenses for other pharmaceuticals, and the like. Modeling module 118 may then apply a first business rule to determine or estimate a financial return, if copay assistance is offered. For example, modeling module 118 may apply one or more of pay as little as, annual cap, monthly cap, per-prescription cap, bridge discount, bridge number, cash discount, and the like. Using the first business rule, modeling module 118 may estimate a patient's post-assistant out-of-pocket cost. The post-assistance out-of-pocket cost may correspond to the patient's financial burden with copay assistance. The post-assistance out-of-pocket cost may be based on one or more of the proposed copay assistance, the patient's deductible, out-of-pocket maximum, copay/co-insurance, out-of-pocket monthly expenses for other pharmaceuticals, and the like. Modeling module 118 may then compare the out-of-pocket sensitivity to the post-assistance out-of-pocket cost to determine or estimate the probability of the patient paying the post-assistance out-of-pocket cost. Based on this information, modeling module 118 may project or estimate a financial return to manufacturer system 106 for offering copay assistance.

In some embodiments, modeling module 118 may repeat the above process for more than one business rule. In this manner, modeling module 118 may generate a plurality of financial return estimates or projections. Modeling module 118 may identify or choose that business rule that optimizes or returns the greatest financial return to manufacturer system 106.

In some embodiments, copay module 116 may generate a copay plan for the user via machine learning modules 120. For example, machine learning module 120 may take, as input, at least data obtained from blocks 208-212, as well as any prior agreement data, to generate, as output, a variable copay plan for the patient. Such variable copay plan may provide how much the manufacturer of the pharmaceutical should pay for each prescription or fill that would maximize at least one of revenue or profit for the manufacturer.

In some embodiments, copay module 116 may continually track patient engagement with the pharmaceutical once the copay plan is generated. For example, copay module 116 may track a fulfillment rate (block 218) of the pharmaceutical. In other words, copay module 116 may track whether the patient is following through on subsequent fills (i.e., refills) following the first fill. Given the prescription information (“RX Written”—block 216) and the fulfillment rate (block 218), copay module 116 may derive the total volume of the pharmaceutical used (“Total Volume Used”—block 220). The total volume of the pharmaceutical used may be fed back into copay module 116 for further analysis. For example, given the total volume used, copay module 116 may adjust the one or more algorithms used by modeling module 118 for generating a variable copay plan. In another example, copay module 116 may leverage the total volume used in training or retraining one or more machine learning models used by one or more machine learning modules 120 for generating a copay plan optimized for one or more of revenue or profit.

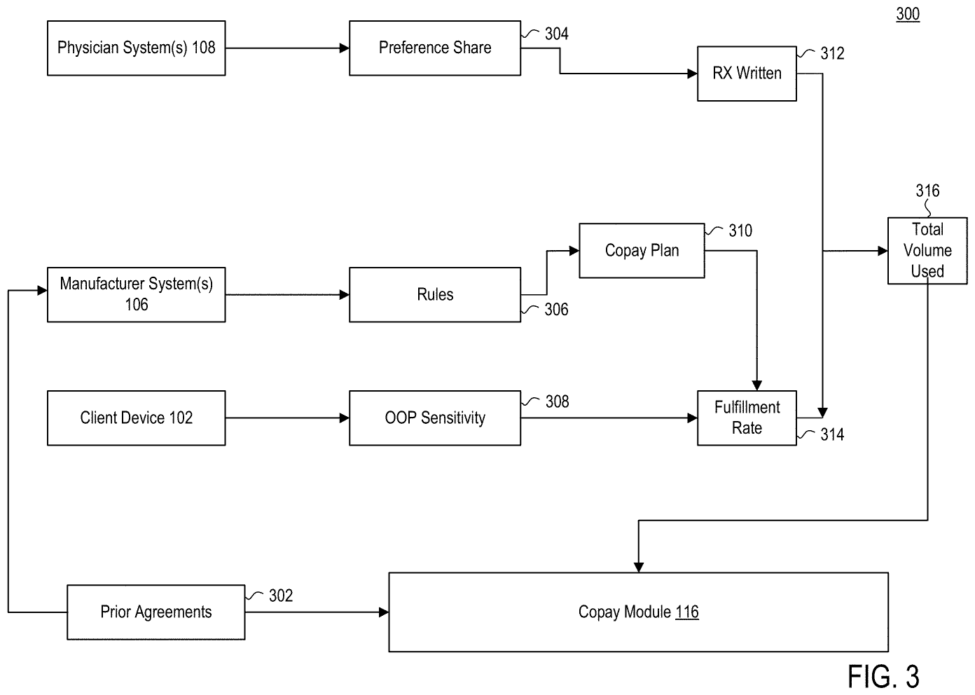

FIG. 3 is a block diagram illustrating an exemplary workflow 300, according to example embodiments. Workflow 300 may be similar to workflow 200 described above in conjunction with FIG. 2. However, workflow 300 may omit payer system 110 from the workflow. Such situation may arise, for example, if the patient is using an out-of-network provider or does not have insurance. As shown, workflow 300 may start with physician system 108. Physician system 108 may want to prescribe a pharmaceutical to a patient (“Preference Share”—block 304). Physician system 108 may generate a prescription (“RX Written”—block 312).

Referring to manufacturer system 106, at block 306, manufacturer system 106 may identify one or more rules. These rules may define the conditions under which copay assistance may pay for a patient's out-of-pocket responsibilities. Exemplary rules may include, but are not limited to, pay as little as, annual cap, monthly cap, per-prescription cap, bridge discount, bridge discount number, and cash discount. The rules at block 306 may be provided to copay module 116 to generate a copay plan (block 310).

Referring to client device 102, at block 308, a patient or user associated with client device 102 may have an out-of-pocket sensitivity. In some embodiments, the out-of-pocket sensitivity may depend on whether this is an initial script or prescription for a pharmaceutical or if this is a refill for the pharmaceutical. For example, for a first fill, patients typically exhibit higher price sensitivities at lower out-of-pocket levels when starting therapy. In some embodiments, copay module 116 may be configured to estimate the various out-of-pocket sensitivities for the patient. For example, copay module 116 may estimate a first out-of-pocket sensitivity for the patient. The first out-of-pocket sensitivity may be associated with the first fill. Copay module 116 may further estimate a second out-of-pocket sensitivity for the patient. The second out-of-pocket sensitivity may be associated with the second fill.

Copay module 116 may generate a copay plan (block 310) for a pharmaceutical based on the out-of-pocket sensitivity of the patient (“OOP Sensitivity”—block 308) and the rules of manufacturer system 106 (“Rules”—block 306). In some embodiments, copay module 116 may further take into consideration any prior agreements (block 302) between the manufacturer and the patient. For example, if the manufacturer offers a rebate on a certain pharmaceutical, copay module 116 may take the rebate amount into consideration.

Copay module 116 may generate a patient journey based on the copay plan. To generate the patient journey, copay module 116 may identify a start date for treatment. For example, copay module 116 may determine which month the user is filling the first script. Such information may assist copay module 116 in determining how the current pharmaceutical may affect the patient's deductible, out-of-pocket maximum, copay, and/or monthly out-of-pocket costs.

To generate the copay plan, modeling module 118 may be configured to identify which business rule to apply based on a plurality of factors. For example, modeling module 118 may estimate a out-of-pocket sensitivity for the patient. In some embodiments, the out-of-pocket sensitivity may be a first fill out-of-pocket sensitivity (i.e., a first sensitivity) or a second fill out-of-pocket sensitivity (i.e., second sensitivity). Using this information, as well as the patient's insurance benefit information, modeling module 118 may generate or estimate the patient's pre-assistance out-of-pocket cost. The pre-assistance out-of-pocket cost may represent the patient's out-of-pocket cost without any copay assistance. The pre-assistance out-of-pocket cost may be based on one or more of the patient's deductible, out-of-pocket maximum, copay/co-insurance, out-of-pocket monthly expenses for other pharmaceuticals, and the like. Modeling module 118 may then apply a first business rule to determine or estimate a financial return, if copay assistance is offered. For example, modeling module 118 may apply one or more of pay as little as, annual cap, monthly cap, per-prescription cap, bridge discount, bridge number, cash discount, and the like. Using the first business rule, modeling module 118 may estimate a patient's post-assistant out-of-pocket cost. The post-assistance out-of-pocket cost may correspond to the patient's financial burden with copay assistance. The post-assistance out-of-pocket cost may be based on one or more of the proposed copay assistance, the patient's deductible, out-of-pocket maximum, copay/co-insurance, out-of-pocket monthly expenses for other pharmaceuticals, and the like. Modeling module 118 may then compare the out-of-pocket sensitivity to the post-assistance out-of-pocket cost to determine or estimate the probability of the patient paying the post-assistance out-of-pocket cost. Based on this information, modeling module 118 may project or estimate a financial return to manufacturer system 106 for offering copay assistance.

In some embodiments, modeling module 118 may repeat the above process for more than one business rule. In this manner, modeling module 118 may generate a plurality of financial return estimates or projections. Modeling module 118 may identify or choose that business rule that optimizes or returns the greatest financial return to manufacturer system 106.

In some embodiments, copay module 116 may generate a copay plan for the user via modeling module 118. For example, modeling module 118 may apply one or more algorithms to at least the data obtained from blocks 306-308, as well as any prior agreements between manufacturer and payer, to generate a variable copay plan for the patient. In some embodiments, modeling module 118 may take into consideration one or more of the estimated first out-of-pocket sensitivity, the estimated second out-of-pocket sensitivity, and/or the estimated out-of-pocket cost with the copay assistance. In some embodiments, copay module 116 may generate a copay plan for the user via machine learning modules 120. For example, machine learning module 120 may take, as input, at least data obtained from blocks 306-308, as well as any prior agreement data, to generate, as output, a variable copay plan for the patient. Such variable copay plan may provide how much the manufacturer of the pharmaceutical should pay for each prescription or fill that would maximize at least one of revenue or profit for the manufacturer.

In some embodiments, copay module 116 may continually track patient engagement with the pharmaceutical once the copay plan is generated. For example, copay module 116 may track a fulfillment rate (block 314) of the pharmaceutical. In other words, copay module 116 may track whether the patient is following through on subsequent fills (i.e., refills) following the first fill. Given the prescription information (“RX Written”—block 312) and the fulfillment rate (block 314), copay module 116 may derive the total volume of the pharmaceutical used (“Total Volume Used—block 316). The total volume of the pharmaceutical used may be fed back into copay module 116 for further analysis. For example, given the total volume used, copay module 116 may adjust the one or more algorithms used by modeling module 118 for generating a variable copay plan. In another example, copay module 116 may leverage the total volume used in training or retraining one or more machine learning models used by one or more machine learning modules 120 for generating a copay plan optimized for one or more of revenue or profit.

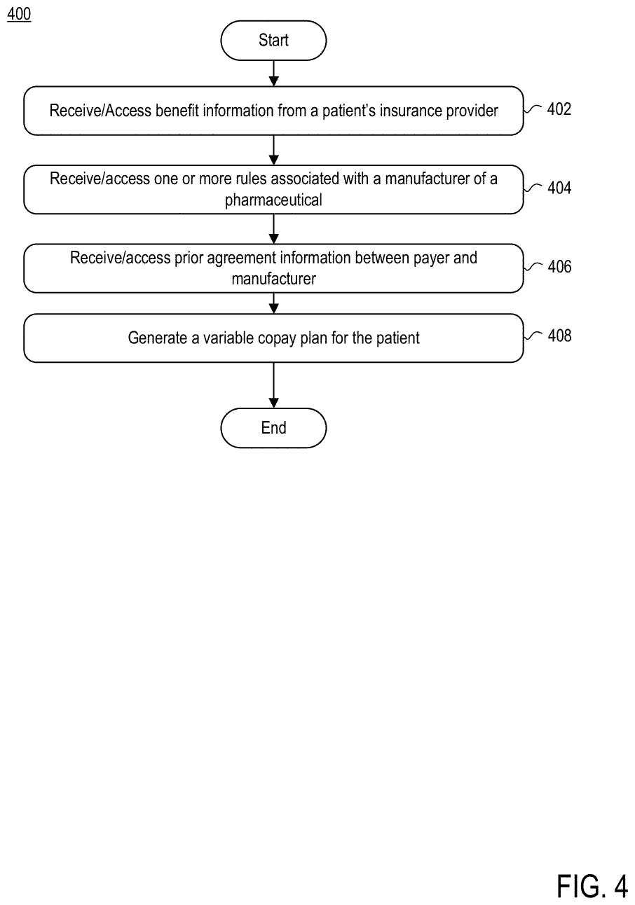

FIG. 4 is a flow diagram illustrating a method 400 of generating a variable copay plan for a patient, according to example embodiments. Method 400 may begin at step 402.

At step 402, organization computing system 104 may receive or access benefit information from the patient's insurance provider. For example, organization computing system 104 may communicate with payer system 110 to obtain the patient's benefits under their insurance program. In some embodiments, the patient's benefits may depend on the type of plan the patient has with payer system 110. For example, a patient may have a HDHP or a non-HDHP. In both plans—HDHP and non-HDHP—the patient has various benefits. Exemplary benefits may include, but are not limited to, an out-of-pocket maximum, a deductible, a copay or coinsurance, and/or a monthly out-of-pocket costs. In some embodiments, these benefits may differ across patients or users, depending on the type of plan the patient or user has subscribed to.

At step 404, organization computing system 104 may receive or access one or more rules associated with a manufacturer of a pharmaceutical to be prescribed to the patient. For example, organization computing system 104 may communicate with manufacturer system 106 to obtain rule information for the manufacturer. The rules may define the conditions under which copay assistance may pay for a patient's out-of-pocket responsibilities. Exemplary rules may include, but are not limited to, pay as little as, annual cap, monthly cap, per-prescription cap, bridge discount, bridge discount number, and cash discount. Modeling module 118 may choose a given rule based on the market or based on an optimization target, as defined by manufacturer system 106.

At step 406, organization computing system 104 may receive or access any prior agreement information (if any) between payer and manufacturer. For example, a payer and manufacturer may come to an agreement regarding a rebate for a pharmaceutical manufactured by manufacturer system 106. Such rebate information may affect the copay plan generated by copay module 116.

At step 408, organization computing system 104 may generate a variable copay plan for the patient. For example, copay module 116 may generate a variable copay plan for the patient based on at least one or more of the benefit information, the one or more rules of the manufacturer, and/or any prior agreement information.

In some embodiments, copay module 116 may generate the variable copay plan via modeling module 118. For example, modeling module 118 may be configured to optimize the various rules for any drug and insurance combination. To do so, modeling module 118 may take a multi-step approach. In some embodiments, modeling module 118 may be configured to estimate the patient out-of-pocket sensitivity for a first prescription fill for a pharmaceutical. In some embodiments, modeling module 118 may be configured to estimate patient out-of-pocket sensitivity for refills for the drug. In some embodiments, modeling module 118 may further model patient insurance. For example, modeling module 118 may take into consideration one or more of the patient's deductible, out-of-pocket maximum, copay or coinsurance, and/or patient out-of-pocket monthly fees for other prescriptions. In some embodiments, modeling module 118 may further model other financial characteristics for the manufacturer. Exemplary financial characteristics may include, but are not limited to, drug list price (e.g., wholesale acquisition cost), cost of goods sold, distribution costs, copay assistance administrative costs, rebate paid to an insurance company, 340B discount, and the like. In some embodiments, modeling module 118 may further model new-patient prescriptions. For example, modeling module 118 may model new-patient prescriptions each calendar month over a year or more. In some embodiments, modeling module 118 may test various combinations of rules. For example, depending on manufacturer input, modeling module 118 may model to optimize for revenue or optimize for profit.

As output, modeling module 118 may generate a table of benefits the provides how much copay a manufacturer will or should cover for each fill of the prescription. In some embodiments, such information may be transparent to the patient.

In some embodiments, copay module 116 may generate the variable copay plan via one or more machine learning modules 120. One or more machine learning module 120 may be optimized or trained to generate a copay program that achieves a certain business rule. For example, machine learning module 120 may be configured to optimize the various rules for any drug and insurance combination. Using a specific example, for a given patient and a given pharmaceutical, machine learning module 120 may generate a first copay program that takes into account one or more of (1) when in the year the patient is starting the pharmaceutical; (2) the patient's deductible; (3) the patient's out-of-pocket maximum; (4) any coinsurance; and (5) any other out-of-pocket expenses for other pharmaceuticals. In some embodiments, machine learning module 120 may further take into account one or more financial characteristics, such as, but not limited to, the list price of the pharmaceutical, cost of goods sold, distribution costs, administrative costs, rebates paid to the patient's payer, and the like.

In some embodiments, manufacturer system 106 may adjust a target goal of machine learning module 120. For example, manufacturer system 106 may specify that machine learning module 120 should generate a copay program that optimizes for revenue. In this matter, machine learning module 120 may adjust one or more weights of a machine learning model to optimize for revenue. Using another example, manufacturer system 106 may specify that machine learning module 120 should generate a copay program that optimizes for profit. In this matter, machine learning module 120 may adjust one or more weights of a machine learning model to optimize for profit.

As output, machine learning modules 120 may generate a table of benefits the provides how much copay a manufacturer will or should cover for each fill of the prescription. In some embodiments, such information may be transparent to the patient.

Once the variable copay plan is generated, when patient purchases a prescription, they may pay a fee that may be offset by a copay amount for their respective fill. For example, when a patient goes to a pharmacy and the pharmacy scans their insurance card, the amount showed as being owed may correspond to the pharmaceutical amount minus any copay assistance for that prescription fill, as defined by the variable copay program. In some embodiments, the patient may have a separate card linked to organization computing system 104. In this manner, when a patient provides a pharmacy with the card, the pharmacy may apply any copay assistance to the price of the pharmaceutical for that prescription fill, as defined by the variable copay program.

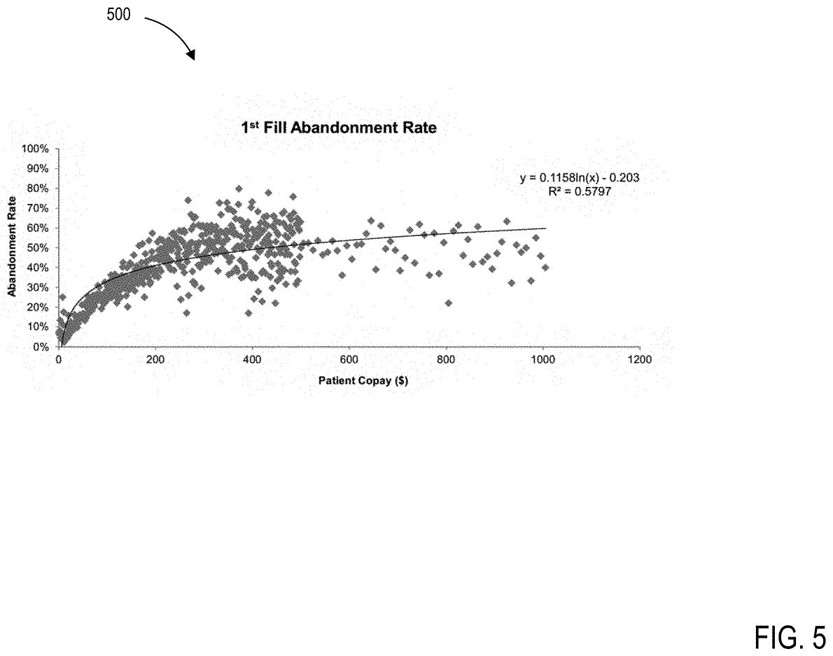

FIG. 5 is a chart 500 that illustrates a first fill abandonment rate, according to example embodiments. As shown in chart 500, patients may exhibit higher price sensitivity at lower out-of-pocket levels when starting therapy for a pharmaceutical.

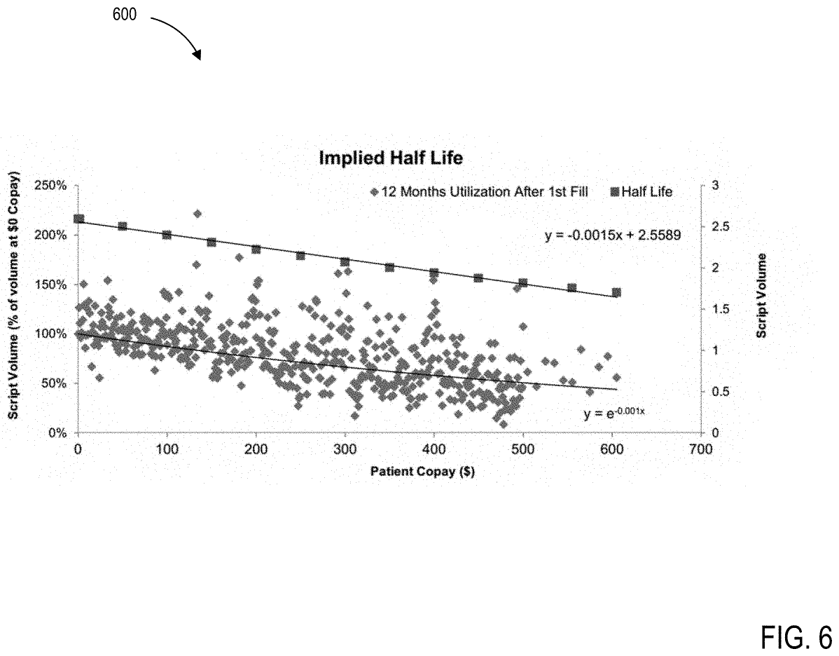

FIG. 6 is a chart 600 that illustrates a pharmaceutical half-life, according to example embodiments. As shown, the half life may be defined as the number of scripts on average for 50% of patients to drop off from therapy. Chart 600 may show the half life at different out-of-pocket levels across a period of twelve months.

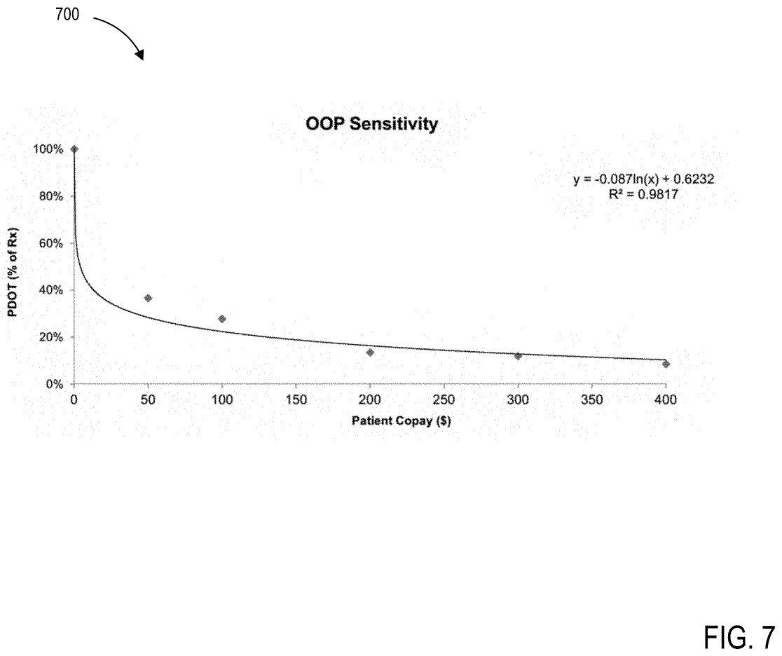

FIG. 7 is a chart 700 that illustrates average patient days on therapy, according to example embodiments. As shown, chart 700 illustrates the first fill rate and attrition rate from a second fill or later fill to estimate an annual patient days on therapy for patients as a function of out-of-pocket sensitivity.

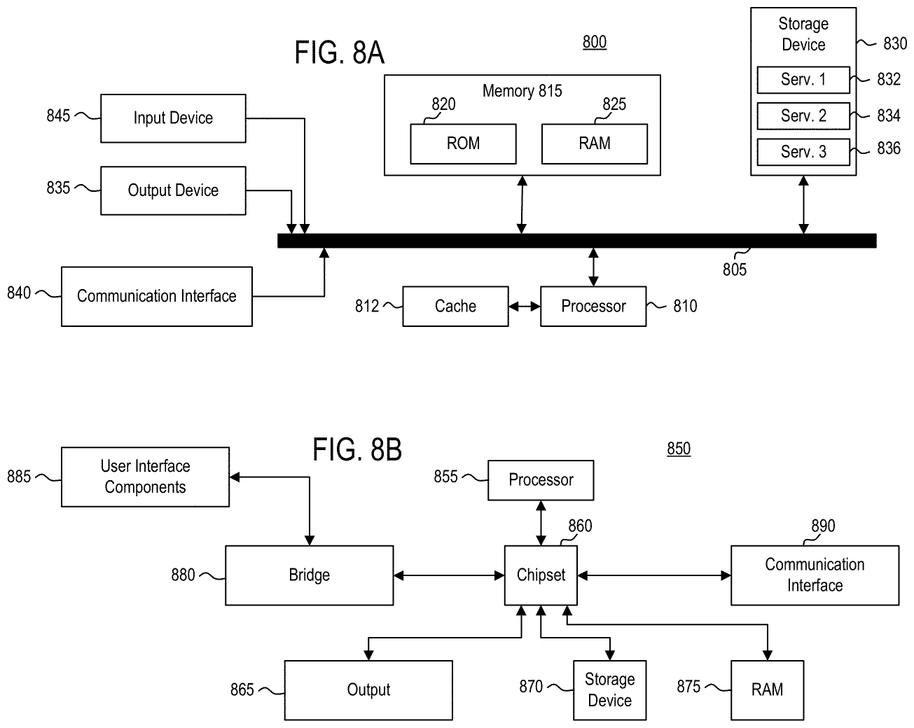

FIG. 8A illustrates an architecture of system bus computing system 800, according to example embodiments. One or more components of system 800 may be in electrical communication with each other using a bus 805. System 800 may include a processor (e.g., one or more CPUs, GPUs or other types of processors) 810 and a system bus 805 that couples various system components including the system memory 815, such as read only memory (ROM) 820 and random access memory (RAM) 825, to processor 810. System 800 can include a cache of high-speed memory connected directly with, in close proximity to, or integrated as part of processor 810. System 800 can copy data from memory 815 and/or storage device 830 to cache 812 for quick access by processor 810. In this way, cache 812 may provide a performance boost that avoids processor 810 delays while waiting for data. These and other modules can control or be configured to control processor 810 to perform various actions. Other system memory 815 may be available for use as well. Memory 815 may include multiple different types of memory with different performance characteristics. Processor 810 may be representative of a single processor or multiple processors. Processor 810 can include one or more of a general purpose processor or a hardware module or software module, such as service 1 832, service 2 834, and service 8 836 stored in storage device 830, configured to control processor 810, as well as a special-purpose processor where software instructions are incorporated into the actual processor design. Processor 810 may essentially be a completely self-contained computing system, containing multiple cores or processors, a bus, memory controller, cache, etc. A multi-core processor may be symmetric or asymmetric.

To enable user interaction with the system 800, an input device 845 which can be any number of input mechanisms, such as a microphone for speech, a touch-sensitive screen for gesture or graphical input, keyboard, mouse, motion input, speech and so forth. An output device 835 (e.g., a display) can also be one or more of a number of output mechanisms known to those of skill in the art. In some instances, multimodal systems can enable a user to provide multiple types of input to communicate with system 800. Communications interface 840 can generally govern and manage the user input and system output. There is no restriction on operating on any particular hardware arrangement and therefore the basic features here may easily be substituted for improved hardware or firmware arrangements as they are developed.

Storage device 830 may be a non-volatile memory and can be a hard disk or other types of computer readable media that can store data that are accessible by a computer, such as magnetic cassettes, flash memory cards, solid state memory devices, digital versatile disks, cartridges, random access memories (RAMs) 825, read only memory (ROM) 820, and hybrids thereof.

Storage device 830 can include services 832, 834, and 836 for controlling the processor 810. Other hardware or software modules are contemplated. Storage device 830 can be connected to system bus 805. In one aspect, a hardware module that performs a particular function can include the software component stored in a computer-readable medium in connection with the necessary hardware components, such as processor 810, bus 805, output device 835 (e.g., a display), and so forth, to carry out the function.

FIG. 8B illustrates a computer system 850 having a chipset architecture, according to example embodiments. Computer system 850 may be an example of computer hardware, software, and firmware that can be used to implement the disclosed technology. System 850 can include one or more processors 855, representative of any number of physically and/or logically distinct resources capable of executing software, firmware, and hardware configured to perform identified computations. One or more processors 855 can communicate with a chipset 860 that can control input to and output from one or more processors 855. In this example, chipset 860 outputs information to output 865, such as a display, and can read and write information to storage device 870, which can include magnetic media, and solid-state media, for example. Chipset 860 can also read data from and write data to storage device 875 (e.g., RAM). A bridge 880 for interfacing with a variety of user interface components 885 can be provided for interfacing with chipset 860. Such user interface components 885 can include a keyboard, a microphone, touch detection and processing circuitry, a pointing device, such as a mouse, and so on. In general, inputs to system 850 can come from any of a variety of sources, machine generated and/or human generated.

Chipset 860 can also interface with one or more communication interfaces 890 that can have different physical interfaces. Such communication interfaces can include interfaces for wired and wireless local area networks, for broadband wireless networks, as well as personal area networks. Some applications of the methods for generating, displaying, and using the GUI disclosed herein can include receiving ordered datasets over the physical interface or be generated by the machine itself by one or more processors 855 analyzing data stored in storage device 870 or 875. Further, the machine can receive inputs from a user through user interface components 885 and execute appropriate functions, such as browsing functions by interpreting these inputs using one or more processors 855.

It can be appreciated that example systems 800 and 850 can have more than one processor 810 or be part of a group or cluster of computing devices networked together to provide greater processing capability.

While the foregoing is directed to embodiments described herein, other and further embodiments may be devised without departing from the basic scope thereof. For example, aspects of the present disclosure may be implemented in hardware or software or a combination of hardware and software. One embodiment described herein may be implemented as a program product for use with a computer system. The program(s) of the program product define functions of the embodiments (including the methods described herein) and can be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed embodiments, are embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A method performed by a computing system, said method comprising:
receiving an indication of a pharmaceutical to be prescribed to a patient;
based on the indication, receiving benefit information from a payer system associated with the patient, the benefit information comprising one or more parameters for an insurance program provided by the payer system to the patient;
receiving, one or more rules associated with a manufacturer system associated with the pharmaceutical to be prescribed to the patient, the one or more rules defining conditions under which copay assistance is used to cover out-of-pocket responsibilities for the patient;
generating a variable copay program for the patient, the variable copay program comprising a variable amount of copay assistance for each fill of the pharmaceutical to be prescribed to the patient; and
generating a table accessible to one or more third party systems, the table comprising the variable copay program.

2. The method of claim 1, wherein generating the variable copay program for the patient comprises:
estimating a first out-of-pocket sensitivity for the patient;
estimating a pre-assistance out-of-pocket cost for the patient, wherein the pre-assistance out-of-pocket cost is a first cost for the patient without copay assistance;
applying a first business rule to the pre-assistance out-of-pocket cost;
estimating a post-assistance out-of-pocket cost for the patient, wherein the post-assistance out-of-pocket cost is a second cost for the patient with copay assistance;
estimating a probability of the patient paying the post-assistance out-of-pocket cost; and
based on the first out-of-pocket sensitivity, the pre-assistance out-of-pocket cost, the first business rule, the post-assistance out-of-pocket cost, and the probability, generating an estimated financial return comparing offering copay assistance versus not offering copay assistance.

3. The method of claim 2, further comprising:
applying a second business rule to the pre-assistance out-of-pocket cost;
estimating a second post-assistance out-of-pocket cost for the patient;
estimating a second probability of the patient paying the second post-assistance out-of-pocket cost;
based on the first out-of-pocket sensitivity, the pre-assistance out-of-pocket cost, the second business rule, the second post-assistance out-of-pocket cost, and the second probability, generating a second estimated financial return comparing offering copay assistance versus not offering copay assistance; and
selecting the first business rule or the second business rule based on which rule yields a larger return.

4. The method of claim 1, wherein generating the variable copay program for the patient comprises:
utilizing one or more machine learning models to optimize profits for the manufacturer system based on the benefit information from the payer system and the one or more rules associated with the manufacturer system.

5. The method of claim 1, wherein generating the variable copay program for the patient comprises:
utilizing one or more machine learning models to optimize revenue for the manufacturer system based on the benefit information from the payer system and the one or more rules associated with the manufacturer system.

6. The method of claim 1, wherein generating the variable copay program for the patient comprises:
estimating an out-of-pocket responsibility for the patient for each prescription fill of the pharmaceutical.

7. The method of claim 1, further comprising:
receiving a second indication of the pharmaceutical being prescribed to a second patient;
based on the indication, receiving second benefit information from a second payer system associated with the second patient, the second benefit information comprising one or more second parameters for a second insurance program provided by the second payer system to the second patient;
receiving, a second set of one or more rules associated with the manufacturer system associated with the pharmaceutical to be prescribed to the patient, the second set of one or more rules defining a second set of conditions under which a second copay assistance is used to cover out-of-pocket responsibilities for the second patient;
generating a second variable copay program for the second patient, the second variable copay program comprising a second variable amount of second copay assistance for each fill of the pharmaceutical to be prescribed to the second patient; and
updating the table accessible to the one or more third party systems, the updated table comprising the second variable copay program.

8. A non-transitory computer readable medium having one or more sequences of instructions, which, when executed by one or more processors, causes a computing system to perform operations comprising:
receiving an indication of a pharmaceutical to be prescribed to a patient;
based on the indication, receiving benefit information from a payer system associated with the patient, the benefit information comprising one or more parameters for an insurance program provided by the payer system to the patient;
receiving one or more rules associated with a manufacturer system associated with the pharmaceutical to be prescribed to the patient, the one or more rules defining conditions under which copay assistance is used to cover out-of-pocket responsibilities for the patient;
generating a variable copay program for the patient, the variable copay program comprising a variable amount of copay assistance for each fill of the pharmaceutical to be prescribed to the patient; and
generating a table accessible to one or more third party systems, the table comprising the variable copay program.

9. The non-transitory computer readable medium of claim 8, further comprising:
receiving prior agreement information between the payer system and the manufacturer system, the prior agreement information comprising rebate information for the pharmaceutical.

10. The non-transitory computer readable medium of claim 8, wherein generating the variable copay program for the patient comprises:
utilizing one or more machine learning models to optimize profits for the manufacturer system based on the benefit information from the payer system and the one or more rules associated with the manufacturer system.

11. The non-transitory computer readable medium of claim 8, wherein generating the variable copay program for the patient comprises:
utilizing one or more machine learning models to optimize revenue for the manufacturer system based on the benefit information from the payer system and the one or more rules associated with the manufacturer system.

12. The non-transitory computer readable medium of claim 8, wherein generating the variable copay program for the patient comprises:
estimating an out-of-pocket responsibility for the patient for each prescription fill of the pharmaceutical.

13. The non-transitory computer readable medium of claim 8, wherein the benefit information comprises one or more of an out-of-pocket maximum, a deductible, a copay or coinsurance, or a monthly out-of-pocket expense for all other pharmaceuticals prescribed to the patient.

14. The non-transitory computer readable medium of claim 8, wherein the one or more rules comprise one or more of pay as little as, an annual cap, a monthly cap, a per-prescription cap, a bridge discount, a bridge discount number, or a cash discount.

15. A system comprising:
one or more processors; and
a memory having programming instructions stored thereon, which, when executed by the one or more processors, causes the system to perform operations, comprising:
receiving an indication of a pharmaceutical to be prescribed to a patient;
based on the indication, receiving benefit information from a payer system associated with the patient, the benefit information comprising one or more parameters for an insurance program provided by the payer system to the patient;
receiving one or more rules associated with a manufacturer system associated with the pharmaceutical to be prescribed to the patient, the one or more rules defining conditions under which copay assistance is used to cover out-of-pocket responsibilities for the patient;
generating a variable copay program for the patient, the variable copay program comprising a variable amount of copay assistance for each fill of the pharmaceutical to be prescribed to the patient; and
generating a table accessible to one or more third party systems, the table comprising the variable copay program.

16. The system of claim 15, wherein the operations further comprise:
receiving prior agreement information between the payer system and the manufacturer system, the prior agreement information comprising rebate information for the pharmaceutical.

17. The system of claim 15, wherein generating the variable copay program for the patient comprises:
utilizing one or more machine learning models to optimize profits for the manufacturer system based on the benefit information from the payer system and the one or more rules associated with the manufacturer system.

18. The system of claim 15, wherein generating the variable copay program for the patient comprises:
utilizing one or more machine learning models to optimize revenue for the manufacturer system based on the benefit information from the payer system and the one or more rules associated with the manufacturer system.

19. The system of claim 15, wherein generating the variable copay program for the patient comprises:
estimating an out-of-pocket responsibility for the patient for each prescription fill of the pharmaceutical.

20. The system of claim 15, wherein the benefit information comprises one or more of an out-of-pocket maximum, a deductible, a copay or coinsurance, or a monthly out-of-pocket expense for all other pharmaceuticals prescribed to the patient and wherein the one or more rules comprise one or more of pay as little as, an annual cap, a monthly cap, a per-prescription cap, a bridge discount, a bridge discount number, or a cash discount.
